(19) United States
(12) Patent Application Publication
(10) Pub. No.: US 2023/0398039 A1
(43) Pub. Date: Dec. 14, 2023

(54) MEDICATION MANAGEMENT DEVICE AND MEDICATION MANAGEMENT SYSTEM

(71) Applicant: H&H CONNECT Co., Ltd., Tokyo (JP)

(72) Inventors: Takuto SAKO, Tokyo (JP); Zhe LIN, Tokyo (JP); Kazumichi BANDO, Tokyo (JP); Satoshi MOCHIZUKI, Yokohama City (JP)

(21) Appl. No.: 18/249,349

(22) PCT Filed: Mar. 26, 2021
(86) PCT No.: PCT/JP2021/012819

§ 371 Date: Apr. 17, 2023

(30) Foreign Application Priority Data
Jan. 25, 2021 (JP) 2021-009772

(51) Int. Cl. A61J 7/04; G16H 20/13; G16H 40/67

(52) U.S. Cl. CPC A61J 7/0436; G16H 20/13; G16H 40/67; A61J 2200/30

(57) ABSTRACT

A medication management device that enables healthcare professionals and their families to immediately ascertain whether a patient has taken the medicine he/she should have taken at the correct time. The present invention with holding unit 12 capable of holding the packaged medicine 32, detection unit 15 capable of detecting changes in the state of holding unit 12, and judgment unit 16 to determine that medicine 32 has been removed when detection unit 15 detects a change in the state of holding unit 12, enables healthcare professionals and their families to immediately ascertain whether a patient has taken the medication he/she should have taken at the correct time.

### TECHNICAL FIELD

The present invention relates to a device and system for managing medication.

### BACKGROUND TECHNOLOGY

Regarding medication, physicians diagnose accurately and prescribe the most appropriate medicines. However, there are cases in which the patient does not take the medication correctly. For example, a mistake in taking the wrong dose may cause side effects, or forgetting to take a dose may fail to cure the patient.

Various initiatives are being taken in medical practice to control the abovementioned cases. For example, “one pack” (Patent Document 1 below), in which medicines to be taken at one time are put in one pouch (sachet), and “medicine calendar” (Patent Document 2 below), which provides a separate sachet containing medicine for each timing of medication, are used.

### PRIOR ART REFERENCE

### Patent Document

[Patent Document 1] Unexamined Patent Publication No. 2011-011753
[Patent Document 2] Utility Model Registration Publication No. 3197075

### SUMMARY OF THE INVENTION

### Problems to be Solved by the Invention

The amount of leftover medicines in Japan is over 100 billion yen annually. The background for failure to take medication correctly differs between outpatients and inpatients.

One of the problems with outpatient medication management is that a high percentage of patients are older adults who live alone, and ascertaining whether they are taking their medication is only possible when their families or healthcare professionals who provide home health care come to check on them or to set the next dose of medication in the medicine calendar. Even if it is ascertained that the medication is being taken, it is not possible to ascertain whether the timing (before, after and between meals) for taking the medication is followed.

One of the problems with inpatient medication management is that although nurses (healthcare professionals) administer medication to patients admitted in hospitals where there are many patients, the nurses often forget to administer medication because they cannot manage the medication correctly. The number of cases where nurses forget to give medicines is very high, and double-checking to prevent it burdens nurses.

Given these issues, the purpose of the present invention is to provide a medication management device and system that enables healthcare professionals and family members to immediately ascertain whether and when a patient has taken the medication.

### Means for Solving the Problems

The medication management device in the first aspect of the present invention is characterized by the following:
A holding unit capable of holding the packaged medicine;
A detection unit capable of detecting the state of the holding unit and changes; and
When the detection unit detects a change in the state of the holding unit, a judgment unit that determines whether the medicine was placed or removed.

More preferably, characterized by:
A PCB on which the timing of use of the medicine is displayed on the surface; and
A holding unit provided for each of the timing displayed on the surface; and
A detection unit provided at a position corresponding to the holding unit on the surface.

More preferably, characterized by:
A judgment unit that also determines the timing at which the medicine is placed or removed based on the position of each detection unit.

More preferably, characterized by:
A holding unit,
Equipped with No. 1 fixation member fixed to the surface; and
An elastic body provided on the No. 1 fixation member, and No. 1 rotation member having a rotation body that is rotatable with respect to the No. 1 fixation member attached to the elastic body; and
The lower end is fixed to the rotation body and rotatable with respect to the No. 1 fixation member and a plate-shaped member pressed against the detection unit provided on the surface by the elastic force of the elastic body in its natural state.

More preferably, characterized by:
A holding unit,
Equipped with a PCB side plate-shaped member fixed to the surface; and
No. 2 fixation member fixed to the PCB side plate-shaped member;
Rotation member 2 provided such that it is rotatable with respect to No. 2 fixation member through the elastic body; and
The lower end side is fixed to the rotation member and rotatable with respect to rotation member 2, and an opening and closing plate-shaped member pressed against the PCB side plate-shaped member by the elastic force of the elastic body in its natural state.

More preferably, characterized by:
A communication control means capable of transmitting information determined by the judgment unit to the outside through a network line.

The medication management system in the second aspect of the present invention is characterized by the following:
A medication management device and a server to receive the information.

More preferably, characterized by:
A terminal capable of receiving the information from the server and notifying the information received.

### Effect of the Invention

The medication management device and system, according to the present invention, enables healthcare professionals and family members to immediately ascertain whether a patient has taken the medication he/she should have taken at the correct time.

### BRIEF DESCRIPTION OF THE DRAWINGS

FIG. 1 A schematic diagram showing the configuration of the medication management system related to embodiment 1 of the present invention.

FIG. 2 A block diagram illustrating the judgment unit, communication control means and battery according to embodiment 1 of the present invention.

FIG. 3 An enlarged perspective view of the holding unit periphery according to embodiment 1 of the present invention.

FIG. 4 A cross-section view a-a of FIG. 3.

FIG. 5 A schematic diagram of a sachet containing the medication.

FIG. 6 A flowchart illustrating an example of the processing by the medication management system according to embodiment 1 of the present invention.

FIG. 7 A flowchart illustrating another example of the processing by the medication management system according to embodiment 1 of the present invention.

FIG. 8 An overall perspective view to explain the configuration of the medication management device related to embodiment 2 of the present invention.

FIG. 9 An enlarged perspective view of the holding unit periphery for embodiment 2 of the present invention.

FIG. 10 A cross-sectional view b-b of FIG. 9.

FIG. 11 A block diagram illustrating the judgment unit, communication control means and battery according to embodiment 2 of the present invention.

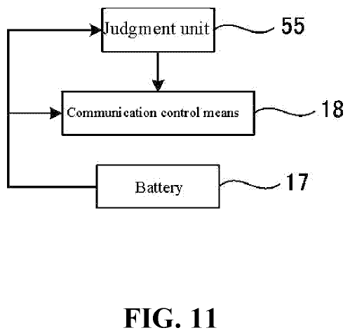

### MODE FOR CARRYING OUT THE INVENTION

The medication management device and system of the present invention will be explained for embodiments using drawings. The phrase “medication” mainly refers to oral medicines in the following embodiments. However, the present invention applies not only to oral medicines but also to the use of medicines in general, such as topical and inhalation medications. Accordingly, although the phrase “sachet” is used in the embodiments, the term applies not only to the sachet but also to the general packaging of the medicine.

### Embodiment 1

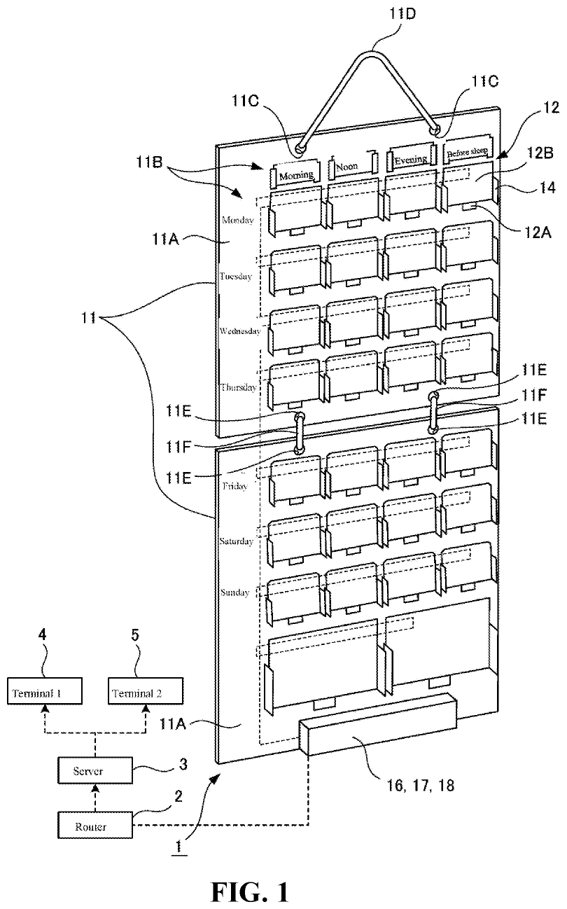

As shown in FIG. 1, according to this embodiment, the medication management system has a medication management device 1, router 2, server 3, No. 1 terminal 4 and No. 2 terminal 5. The following describes the configuration of medication management device 1.

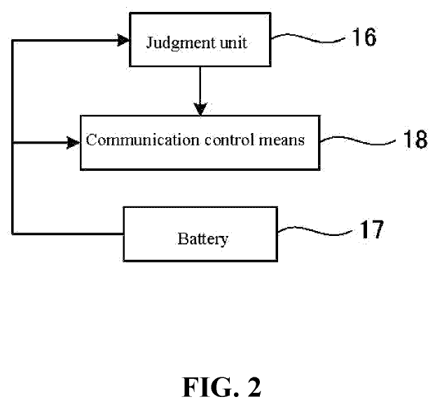

The main configuration of medication management device 1, as shown in FIGS. 1 to 4, consists of PCB 11, holding unit 12, through hole 13, side plate 14, detection unit 15, judgment unit 16, battery 17, and communication control means 18. Of these, holding unit 12, through hole 13, side plate 14, detection unit 15, judgment unit 16, battery 17, and communication control means 18 are all attached to PCB 11.

PCB 11 is a flat plate. PCB 11 may be one plate, or as shown in FIG. 1, it may consist of 2 plates connected vertically by 2 string members 11F that pass through and are fixed in each hole 11E.

Display unit 11B for displaying the timing of medication is provided on surface 11A of PCB 11. In FIG. 1, the day of the week (Monday to Sunday) is displayed as the upper end in the vertical axis on the horizontal end of PCB surface 11A (left side), and timing (morning, noon, evening and before sleeping) of medication is displayed as the horizontal axis in the vertical direction of PCB surface 11A. This is display unit 11B.

Incidentally, this is only one example; the display of display unit 11B may be changed. For example, the date may be displayed as the vertical axis, the vertical axis may be timing, and the horizontal axis may be the day of the week or date.

In any case, display unit 11B, by providing the vertical axis and horizontal axis, displays the timing of medication (day of the week and timing, or date and timing) on PCB surface 11A.

Further, PCB 11 is installed, so the vertical axis is in the vertical direction. Therefore, at the upper end of the vertical direction of PCB 11 (on the horizontal axis), holes 11C are formed in 2 places symmetrically on both sides, ends of string members 11D are attached to the 2 holes 11C, respectively, and PCB 11 is installed by hanging string member 11D at any place.

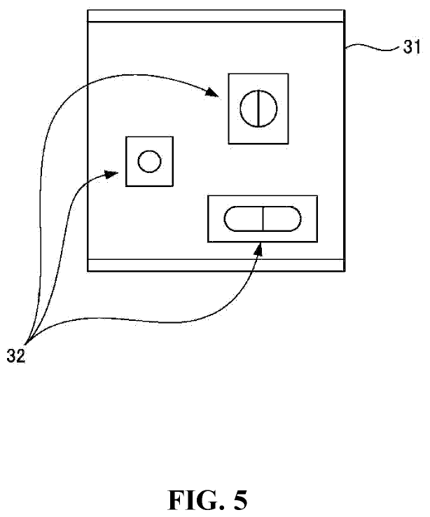

Holding unit 12 includes an opening and closing means 12A and plate-shaped member 12B, and holds sachet 31 containing medicine 32, as shown in FIG. 5. A plurality of holding units 12 are provided on PCB surface 11A. More specifically, the units are provided at all positions (vertical and horizontal axes), indicating the medication timing (such as Monday morning or Tuesday noon) displayed on display unit 11B on PCB surface 11A. In FIG. 1, the units are provided at 28 locations in 7 rows along the vertical axis direction (vertical direction) and 4 rows in the transverse axis direction (horizontal direction). Further, as shown in FIG. 1, one or more holding units 12 may be provided in a position other than the position indicating the timing of the medication (vertical downward direction).

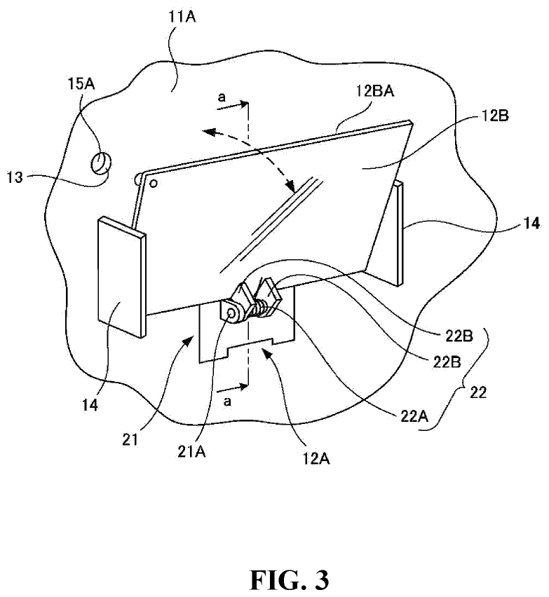

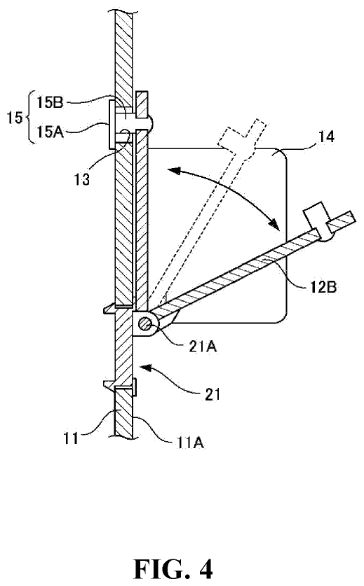

As shown in FIG. 3 and FIG. 4, opening and closing means 12A includes fixation member 21 and rotation member 22. Fixation member 21, fixed to PCB 11, has cylindrical shaft 21A extending in the transverse axis (horizontal) direction on the PCB surface 11A side. Rotation member 22 has elastic body 22A, such as a spring provided on shaft 21A of fixation member 21 and 2 rotation plates (rotation body) 22B fixed to elastic body 22A that is rotatable with respect to fixation member 21.

Lower end side of plate-shaped member 12B is fixed to 2 numbers of rotation body 22B, rotatable with respect to shaft 21A (see the dashed line arrow in FIG. 3). In its natural state, the elastic force of elastic body 22A presses plate-shaped member 12B against PCB surface 11A. Plate-shaped member 12B should be made of a transparent or translucent material; that is, selecting a material such that PCB surface 11A can be seen through plate-shaped member 12B (such as transparent plastic) is preferable.

The holding unit 12 can be made to hold by manually rotating and tilting plate-shaped member 12B in a direction away from PCB surface 11A and placing packaged medicine 32 in sachet 31 between plate-shaped member 12B and PCB surface 11A. “Hold”, in other words, refers to a state in which packaged medicine 32 in sachet 31 is pressed by plate-shaped member 12B to PCB surface 11A.

Further, manually removing packaged medicine 32 in sachet 31 held in holding unit 12 makes it possible to take out medicine 32 packaged in sachet 31. Alternatively, manually rotating and tilting plate-shaped member 12B in a direction away from PCB surface 11A makes it possible to take out packaged medicine 32 in sachet 31.

Side plate 14 is a flat plate fixed vertically to PCB surface 11A above the transverse axis direction (horizontal direction) at the end positions of each holding unit 12 on PCB surface 11A, or more specifically, at the positions sliding against the above the transverse axis direction (horizontal direction) at ends of each plate-shaped member 12B on PCB surface 11A.

Through hole 13 is formed to penetrate in the thickness direction of PCB 11 for each position corresponding to each holding unit 12 on PCB surface 11A, or more specifically, for each position where each plate-shaped member 12B contacts (by the elastic force of elastic body 22A).

Detection unit 15 includes electrode PCB 15A, and conductive rubber 15B is a contact type sensor (membrane switch) for detecting the contact and non-contact states between electrode PCB 15 and conductive rubber 15B. Each electrode PCB 15A is provided on rear surface 11G of PCB 11 to close through hole 13; each conductive rubber 15B is provided on rear surface 12BA of the plate-shaped member 12B at a position corresponding to electrode PCB 15A.

When plate-shaped member 12B is pressed (in contact) against PCB surface 11A, which is the natural state, electrode PCB 15A and conductive rubber 15B are in a contact state. When plate-shaped member 12B is separated from PCB surface 11A, electrode PCB 15A and conductive rubber 15B are in a non-contact state.

Judgment unit 16 is provided at any position of PCB 11 (other than the position indicating the timing of medication described above), and it is judged that packaged medicine 32 in sachet 31 has been removed when detection unit 15 detects a change from a non-contact state to a contact state.

When sachet 31 containing medicine 32 is held in holding unit 12, sachet 31 is sandwiched between PCB surface 11A and rear surface 12BA and plate-shaped member 12B is separated from PCB surface 11A and electrode PCB 15A and conductive rubber 15B are in a non-contact state.

In a state where sachet 31 is not held, plate-shaped member 12B contacts PCB surface 11A by the elastic force of elastic body 22A, and electrode PCB 15A and conductive rubber 15B are in a contact state. Judgment unit 16 determines that sachet 31 has been removed when detection unit 15 detects the change from a non-contact state to a contact state.

Judgment unit 16 may also determine when packaged medicine 32 in sachet 31 is taken out by determining which detection unit 15 among detection units 15 provided at each position that indicates the timing of taking medicine has changed from a non-contact state to a contact state (example packaged medicine 32 in sachet 31 was taken out on Monday morning). This also allows judging if the patient has made a mistake in taking medicine.

Similarly, judgment unit 16 also determines the placement and timing of packaged medicine 32 in sachet 31. When detection unit 15 detects a change from a contact state to a non-contact state, judgment unit 16 determines that packaged medicine 32 in sachet 31 has been placed. This prevents the patient from forgetting to set the medicine (for example, medicine that should be taken tomorrow has not yet been placed in the medicine calendar).

Communication control means 18 is provided at any position of PCB 11 (other than the position indicating the timing of medication described above), and the information (hereinafter, described as judgment information) determined by judgment unit 16 can be transmitted to the outside through a network line.

Battery 17 is provided at any position of PCB 11 (other than the position indicating the timing of medication described above). It supplies power for the operation of detection unit 15, judgment unit 16, and communication control means 18.

The above describes the configuration of the medication management device 1. The following describes the configuration of the medication management system according to this embodiment.

No. 1 terminal 4, shown in FIG. 1, refers to a PC, smartphone, or tablet PC owned by a healthcare professional, and No. 2 terminal 5 refers to a PC, smartphone, or tablet PC owned by a family member of the patient. However, although this embodiment assumes that one No. 1 terminal 4 and one No. 2 terminal 5 are provided, the present invention is not limited to this, multiple numbers of No. 1 terminal 4 and No. 2 terminal 5 may be provided, respectively, or No. 1 terminal 4 or No. 2 terminal 5 may not be provided. The following will be described assuming that No. 1 terminal 4 and No. 2 terminal 5 are provided.

Medication management device 1 through the communication control means 18 of medication management device 1 is capable of communicating with router 2 by Wi-Fi (or LAN) and transmitting information capable of generating the display of the No. 1 terminal 4 and No. 2 terminal 5. Also, router 2 can communicate with server 3 through the Internet.

Server 3 can communicate with router 2, No. 1 terminal 4 and No. 2 terminal 5 through the Internet. When the judgment information (including the timing at which the packaged medicine 32 in sachet 31 is removed) of judgment unit 16 is received from the medication management device 1 through router 2, the judgment information is stored and transmitted to No. 1 terminal 4 and No. 2 terminal 5.

No. 1 terminal 4 and No. 2 terminal 5 can receive the judgment information from judgment unit 16 and be notified by a screen display or audio. If judgment information is not received at the medication timing or if the medication timing is incorrect, a push notification (alert notification) may be provided by a screen display or audio.

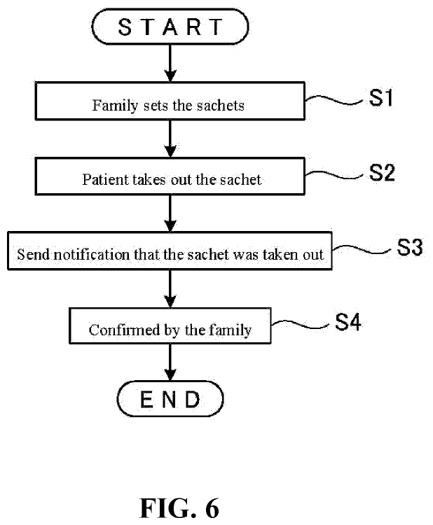

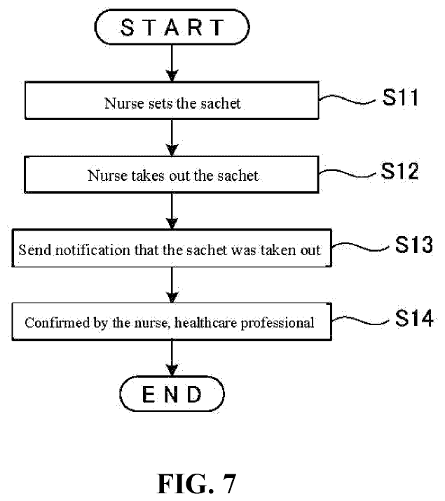

The above describes the configuration of the medication management system according to this embodiment. An example of a process using the medication management system according to this embodiment is described with reference to flowcharts in FIGS. 6 and 7.

FIG. 6 is a flowchart for the case where an outpatient takes the medication at home.

In step S1, a family member of the patient or healthcare professional sets (places) sachet 31 in holding unit 12 of medication management device 1.

In step S2, sachet 31 is removed from holding unit 12 for the patient to take the medication.

In step S3, detection unit 15 is in a contact state by step S2. Judgment unit 16 determines that sachet 31 has been removed from holding unit 12, and the judgment information is transmitted to server 3 through router 2 by communication control means 18. Further, the judgment information is stored in server 3 and transmitted from server 3 to No. 1 terminal 4 and No. 2 terminal 5.

In Step S4, the above-described judgment information displayed on No. 1 terminal 4 and No. 2 terminal 5 is viewed by the family of the patient and healthcare professionals, respectively, to confirm whether sachet 31 was removed from holding unit 12 at the predetermined timing for administration of the medicine, that is, whether the patient took medicine correctly.

If the patient is not taking medicine, it can be confirmed by the family of the patient and the healthcare professional. If the abovementioned judgment information does not reach No. 1 terminal 4 and No. 2 terminal 5 at the predetermined timing of medication, that is, if there are no steps S3 and S4, the family of the patient and the healthcare professional can assume that the patient is not taking medicine (or confirm by an alert notification).

These instructions are for outpatients taking medication at home.

FIG. 7 is a flowchart for the case where an inpatient takes medicine at a hospital.

In step S11, the nurse (healthcare professional) sets (places) sachet 31 in holding unit 12 of medication management device 1.

In step S12, sachet 31 is removed from holding unit 12 by the nurse (healthcare professional) to administer medication to the patient.

In step S13, detection unit 15 is in a contact state from the non-contact state by step S12. Judgment unit 16 determines that sachet 31 has been removed from holding unit 12, and the judgment information is transmitted to server 3 through router 2 by communication control means 18. Further, the judgment information is stored in server 3 and transmitted from server 3 to the No. 1 terminal 4.

In step S14, the nurse (healthcare professional) in charge of medication administration sees the abovementioned judgment information displayed on No. 1 terminal 4 to check whether sachet 31 was removed from holding unit 12 at the predetermined timing of medication administration, that is, whether the patient took medicine.

If the patient is not taking medicine, it can be confirmed by the healthcare professional in charge of medication management. If the abovementioned judgment information does not reach No. 1 terminal 4 at the predetermined timing of medication, that is, if there are no steps S13, S14, the family of the patient and the healthcare professional can consider that the patient is not taking medicine (or confirm by an alert notification).

Further, even in the case of FIG. 6, the judgment information may be transmitted to No. 1 terminal 4, owned by the healthcare professional, and to No. 2 terminal 5, owned by the patient's family. These are the instructions for inpatients taking their medications in the hospital.

In this embodiment, the judgment information from judgment unit 16 of medication management device 1 is transmitted by communication control means 18 to No. 1 terminal 4 and No. 2 terminal 5 through router 2 and server 3; however, the present invention is not limited to this. The specification may be transmitted from the communication control means 18 to server 3 using the Internet or directly from communication control means 18 to No. 1 terminal 4 and No. 2 terminal 5 using the Internet.

In this embodiment, detection unit 15 is assumed to be a contact-type sensor, such as a membrane that detects contact and non-contact; the present invention is not limited to this, and other modes may be used. Detection unit 15 need only be capable of detecting the state and changes of holding unit 12. An example is shown in embodiment 2, described later.

According to this embodiment, since the system is equipped with holding unit 12 capable of holding packaged medicine 32, detection unit 15 capable of detecting the state and changes of holding unit 12, and detection unit 15 when detecting a change in the state of holding unit 12, and judgment unit 16 that judges whether medicine 32 has been placed or removed, it is possible to check if the patient has taken medicine automatically, and also if the patient has forgotten to set the medicine.

According to this embodiment, since PCB 11 is provided with surface 11A on which the timing for using medicine 32 is displayed, holding unit 12 is provided for each medication timing displayed on surface 11A, and detection unit 15 is provided at a position corresponding to holding unit 12 on surface 11A, it is easier for the patient to take the medication at the specified timing.

According to this embodiment, based on the position of each detection unit 15, judgment unit 16 also determines the timing at which medicine 32 is placed or removed, so the timing of taking medicine by the patient can be automatically checked, improving convenience.

According to this embodiment, since the invention is equipped with holding unit 12 having fixation member 21 fixed to surface 11A, rotation member 22 fixed to fixation member 21 having an elastic force toward surface 11A side of the circumferential direction of fixation member 21, plate-shaped member 12B, fixed to rotation member 22 and movable in the circumferential direction with respect to fixation member 21, which in the natural state is pressed with an elastic force against detection unit 15 that is provided on surface 11A, it makes detecting contact and non-contact of detection unit 15 easy and can hold sachet 31 of various sizes (or thicknesses) with a simple structure.

According to this embodiment, since the invention is equipped with a medication management device 1 having communication control means 18 capable of transmitting the judgment information of judgment unit 16 through the network line, server 3 to receive the judgment information of judgment unit 16 from communication control means 18, terminals 4 and 5 capable of receiving information from the server and notifying the received information, healthcare professionals and family members of the patient who possess terminals 4 and 5 can immediately confirm whether and when the patient has taken the medication, or whether and when the patient has forgotten to set the medicine.

According to this embodiment, since the detection unit 15 is a contact type sensor, even if the sachet 31 and plate-shaped member 12B is transparent or semi-transparent can be supported; for example, the detection failure is less compared to the infrared sensor. If the plate-shaped member 12B is made transparent or translucent, it is easier for the patient to visually recognize whether sachet 31 is set in each holding unit 12. If the power consumption is always high, as in the case of devices infrared sensors, an AC power supply or power outlet is required, which limits the installation location and conditions. However, this invention uses contact-type sensors and requires only a DC power supply or battery, and installation locations and conditions are not limited. Also, this invention can be made at a low cost.

### Embodiment 2

The medication management device of this embodiment is a partial modification of medication management device 1 of embodiment 1. Hereinafter, the portion that overlaps with embodiment 1 is omitted to the possible extent and described with reference to the parts that are different.

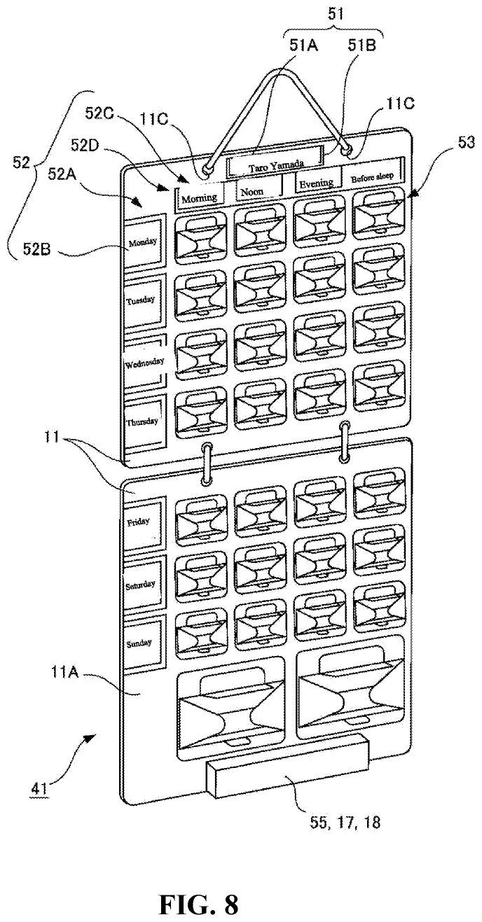

As shown in FIG. 8, the medication management device 41 has name display unit 51, display unit 52 instead of display unit 11B of medication management device 1 according to embodiment 1, holding unit 53 instead of holding unit 12, detection unit 54 instead of detection unit 15, and judgment unit 55 instead of judgment unit 16. Side plate 14 of the medication management device 1, according to embodiment 1, is deleted.

Name display unit 51 is to display the patient's name on the PCB surface 11A and includes name label 51A and label insertion unit 51B.

Name label 51A is a card with the name of the patient. Label insertion unit 51B is provided on PCB surface 11A; for example, at the upper-end portion (between the two holes 11C) of the vertical direction, name label 51A can be inserted and removed, and name label 51A that is inserted can be held.

Display unit 52 displays the timing of medication and is equipped with vertical axis card 52A, vertical axis card insertion unit 52B, horizontal axis card 52C, and horizontal axis card insertion unit 52D.

Vertical axis card 52A, for example, is a card with the day of the week, and a plurality of cards are provided. A plurality of vertical axis card insertion unit 52B is provided on the horizontal end side (left side) of PCB surface 11A, and each vertical axis card 52A can be inserted and removed from the vertical axis card insertion unit 52B, which can hold the inserted vertical axis card 52A. This means that each vertical axis card 52A is held in each vertical axis card insertion unit 52B, corresponding to the vertical axis of Embodiment 1.

Horizontal axis card 52C, for example, is a card describing the medication timing, and a plurality of cards are provided. A plurality of horizontal axis card insertion unit 52D is provided on the upper end of the vertical direction of PCB surface 11A. Each horizontal axis card 52C can be inserted and removed from the horizontal axis card insertion unit 52D, which can hold the inserted horizontal axis card 52C. This means that each horizontal axis card 52C is held in each horizontal axis card insertion unit 52D, corresponding to the horizontal axis of Embodiment 1.

The notation of vertical axis card 52A and horizontal axis card 52C is just one example, and these displays may be changed. For example, the date may be displayed on vertical axis card 52A, or the display of vertical axis card 52A may be the timing, and the display of horizontal axis card 52C may be a day of the week or date.

In any case, display unit 52 displays the timing of medication (day of the week and timing, or date and timing) on PCB surface 11A.

A plurality of holding units 53 are provided on PCB surface 11A. More specifically, the holding unit is provided at all positions indicating the timing of medication on PCB surface 11A, respectively.

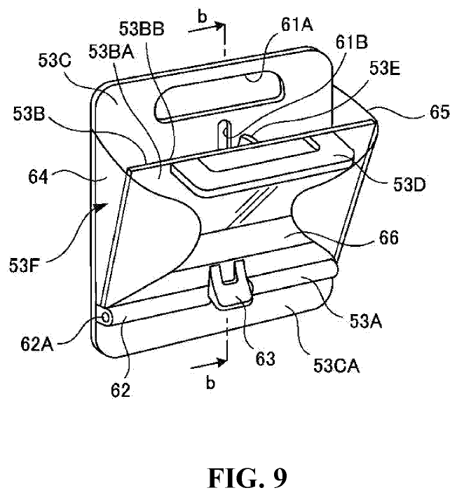

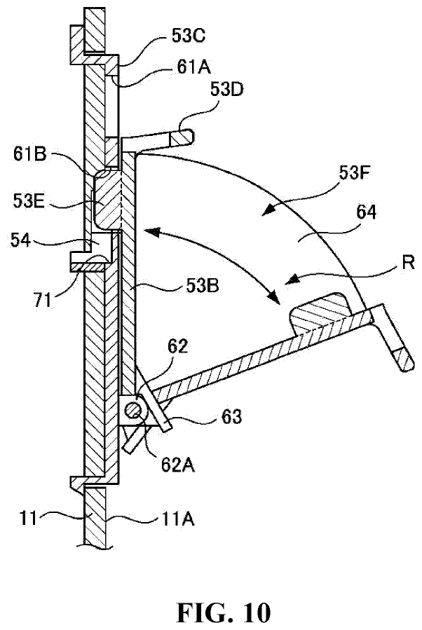

As shown in FIGS. 9 and 10, each holding unit 53 is equipped with opening and closing means 53A, opening and closing plate-shaped member 53B, PCB side plate-shaped member 53C, handle 53D, insertion plate body 53E and side cover 53F.

PCB side plate-shaped member 53C is a flat plate that is fixed at all positions indicating the timing of medication on PCB surface 11A, the vertical direction on the upper-end side, with No. 1 opening 61A penetrating in the thickness direction at the vertical upper end and No. 2 opening 61B penetrating in the thickness direction at the horizontal center. No. 1 opening 61A, is horizontally extended, and No. 2 opening 61B, is vertically extended. Incidentally, if any part of holding unit 53 fails, PCB side plate-shaped member 53C can be removed from the PCB surface 11A.

Opening and closing means 53A includes fixation member 62 and rotation member 63. Fixation member 62 is fixed to PCB side plate-shaped member 53C and has cylindrical shaft 62A extending in the horizontal direction on surface 53CA of PCB side plate-shaped member 53C. Rotation member 63 is rotatable with respect to shaft 62A through an elastic body (such as a spring) provided inside, which is not shown in the diagram.

The lower end side of opening and closing plate-shaped member 53B is fixed to rotation member 63 and is rotatable with respect to shaft 62A. In the natural state, the elastic force of the elastic body in rotation member 63 presses opening and closing plate-shaped member 53B against PCB side plate-shaped member 53C. A transparent or translucent material is used for opening and closing plate-shaped member 53B; in other words, a material through which PCB side plate-shaped member 53C can be seen through the opening and closing plate-shaped member 53B (for example, transparent plastic).

Handle 53D is fixed to the upper end (vertical upper end in the natural state) of surface 53BA side of opening and closing plate-shaped member 53B, which can be held by a human hand.

Insertion plate body 53E is fixed at the center of rear surface 53BB side in the horizontal direction of opening and closing plate-shaped member 53B. It can be inserted into and removed from No. 2 opening 61B as its shape and fixed position corresponding to No. 2 opening 61B. Insertion plate body 53E may be integrally molded with opening and closing plate-shaped member 53B or molded separately and fixed to opening and closing plate-shaped member 53B with an adhesive or other means.

Side cover 53F includes side cover plates 64, 65 and crosslink 66. Thin sheets are used for all these parts; polypropylene or other plastic sheets are preferred. One end of both side cover plates 64, 65 is fixed to the horizontal ends of PCB side plate-shaped member 53C, and the other ends are connected by crosslink 66.

Packaged medicine 32 in sachet 31 can be held between the separated opening and closing plate-shaped member 53B and PCB side plate-shaped member 53C of holding unit 53 by grasping handle 53D by hand and pulling toward the front and tilting the opening and closing plate-shaped member 53B so that it rotates away from the surface of PCB side plate-shaped member 53C. “Hold” herein refers to a state in which packaged medicine 32 in sachet 31 is pressed against PCB side plate-shaped member 53C by opening and closing plate-shaped member 53B.

Further, manually removing packaged medicine 32 in sachet 31 held in holding unit 53 makes it possible to take out medicine 32 packaged in sachet 31. Alternatively, manually rotating and tilting opening and closing plate-shaped member 53B in a direction away from PCB side plate-shaped member 53C makes it possible to take out packaged medicine 32 in sachet 31.

Side cover 53F follows the opening and closing (contact and separation with PCB side plate-shaped member 53C) operation of opening and closing plate-shaped member 53B, specifically when opening and closing plate-shaped member 53B is open, that is, separated from PCB side plate-shaped member 53C, side cover 53F covers both ends along the horizontal direction of opening and closing plate-shaped member 53B and PCB side plate-shaped member 53C forming chamber R between opening and closing plate-shaped member 53B and PCB side plate-shaped member 53C. Packaged medicine 32 in sachet 31 can be held in chamber R.

Detection unit 54, a photosensor is fixed with respect to a part of the edge of No. 2 opening 62B in PCB side plate-shaped member 53C, more particularly with respect to recess 71 formed in the part facing the inside of No. 2 opening 62B, preferably a dust-proof transmission type photosensor.

This allows detection unit 54 to detect the state in which insertion plate body 53E is in the inserted state (hereinafter, inserted state) and not inserted state (hereinafter, non-inserted state) with respect to No. 2 opening 62B.

An opaque material must be used for insertion plate body 53E to increase the detection accuracy of detection unit 54. However, when integrally molding insertion plate body 53E with opening and closing plate-shaped member 53B as previously described, if opening and closing plate-shaped member 53B is made of a transparent or translucent material, insertion plate body 53E will also be made of a transparent or translucent material, and the detection accuracy by detection unit 54 will decrease. In such a case, it is necessary to increase the detection accuracy of detection unit 54 by pasting a seal on the surface of insertion plate body 53E that faces detection unit 54.

Judgment unit 55 is provided at any arbitrary position (other than the position indicating the timing of medication described above) of PCB 11; when detection unit 54 detects the inserted state, it is determined that packaged medicine 32 in sachet 31 has been removed.

That is when sachet 31 containing medicine 32 is held in holding unit 53, insertion plate body 53E is in the non-inserted state since sachet 31 is in a sandwiched state between No. 2 opening 62B and insertion plate body 53E.

When sachet 31 is not held, the elastic force of the elastic body in rotation member 63 causes insertion plate body 53E to be in the inserted state. Judgment unit 55 determines that sachet 31 has been removed when detection unit 54 detects a change from a non-inserted state to inserted state.

Judgment unit 55 may also determine when packaged medicine 32 in sachet 31 is taken out by determining which detection unit 54 provided at each position indicates the timing of taking medicine has changed from a non-inserted state to a contact state. This also allows judging if the patient has made a mistake in taking medicine.

The above describes the configuration of medication management device 41 according to this embodiment.

According to this embodiment, holding unit 53 is unitized (holding section 53 itself completes the configuration for holding packaged medicine 32 without using PCB surface 11A as in embodiment 1). When holding unit 53 fails, the unit can be easily removed from PCB 11 and replaced with a new holding unit 53, thus reducing the man-hours required for handling the failure compared to embodiment 1.

According to this embodiment, detection unit 54 is a photo sensor, compared with embodiment 1 using detection unit 15 for determining contact and non-contact, thereby improving detection accuracy and durability since there is no friction with the detection body.

According to this embodiment, insertion plate body 53E is not only used for detection by detection unit 54 but also as a presser when holding packaged medicine 32 in sachet 31. As a result, sachet 31 can be prevented from falling forward due to its self-weight, for example, when a vertically long sachet 31 is being held.

### INDUSTRIAL APPLICABILITY

The present invention is suitable as a device and system for the management of medication.

### EXPLANATION OF THE REFERENCE NUMERALS

1, 41: Medication management device
2: Router
3: Server
4: No. 1 Terminal
5: No. 2 Terminal
11: PCB
11A: PCB surface
11B, 52: Display unit
11C: Hole
11D: String member
11E: Hole
11F: String member
11G: PCB rear surface
12, 53: Holding unit
12A: Opening and closing means
12B: Plate-shaped member
13: Through hole
14: Partition plate
15, 54: Detection unit
15A: Electrode PCB
15B: Conductive rubber8
16: Judgment unit
17: Battery
18: Communication control means
21, 62: Fixation member
21A, 62A: Shaft
22, 63: Rotation member
22A: Elastic body
22B: Rotation plate
31: Sachet
32: Medicine
51: Name display unit
51A: Name label
51B: Label insertion unit
52A: Vertical axis card
52B: Vertical axis card insertion unit
52C: Horizontal axis card
52D: Horizontal axis card insertion unit
53A: Opening and closing means
53B: Opening and closing plate-shaped member
53C: PCB side plate-shaped member
53D: Handle
53E: Insertion plate
53F: Side cover
61A, 61B: Opening
64, 65: Side cover plate
66: Crosslink
71: Recess

1. A medication management device characterized by a holding unit capable of holding the packaged medicine; and
a detection unit capable of detecting the state of the holding unit and changes; and
when the detection unit detects a change in the state of the holding unit, a judgment unit that determines whether the medicine was placed or removed.

2. A medication management device, according to claim 1 characterized by a PCB on which the timing of use of the medicine is displayed on the surface; and
a holding unit provided for each of the timing displayed on the surface; and
a detection unit provided at a position corresponding to the holding unit on the surface.

3. A medication management device, according to claim 1 characterized by a judgment unit that also determines the timing at which the medicine is placed or removed based on the position of each detection unit.

4. A medication management device, according to claim 2 characterized by a holding unit,
equipped with No. 1 fixation member fixed to the surface; and
an elastic body provided on No. 1 fixation member, and No. 1 rotation member having a rotation body that is rotatable with respect to No. 1 fixation member attached to the elastic body; and
the lower end is fixed to the rotation body and rotatable with respect to the No. 1 fixation member and a plate-shaped member pressed against the detection unit provided on the surface by the elastic force of the elastic body in its natural state.

5. A medication management device, according to claim 2 characterized by a holding unit,
equipped with a PCB side plate-shaped member fixed to the surface; and
No. 2 fixation member fixed to the PCB side plate-shaped member; and
rotation member 2 provided such that it is rotatable with respect to No. 2 fixation member through the elastic body;
the lower end side is fixed to the rotation member and rotatable with respect to rotation member 2, and opening and closing plate-shaped member is pressed against the PCB side plate-shaped member by the elastic force of the elastic body in its natural state.

6. A medication management device, according to claim 1 characterized by a communication control means that is capable of transmitting information determined by the judgment unit to the outside through a network line.

7. A medication management system, according to claim 6 characterized by a medication management device and a server to receive the information.

8. A medication management system, according to claim 7, characterized by a terminal capable of receiving the information from the server and notifying the information received.
